(19) United States
(12) Patent Application Publication
(10) Pub. No.: US 2010/0291707 A1
(43) Pub. Date: Nov. 18, 2010

(54) Multiplexed Scanometric Assay for Target Molecules

(75) Inventors: Chad A. Mirkin, Wilmette, IL (US); Dongwoo Kim, Kyungsangnamdo (KR); Weston L. Daniel, Evanston, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: NORTHWESTERN UNIVERSITY, Evanston, IL (US)

(21) Appl. No.: 12/770,488

(22) Filed: Apr. 29, 2010

Related U.S. Application Data
(60) Provisional application No. 61/173,874, filed on Apr. 29, 2009.

(51) Int. Cl. G01N 33/50

(52) U.S. Cl. 436/501

(57) ABSTRACT

The present invention is directed to compositions and methods of use of a functionalized nanoparticle having a catalytic metal deposit.

### CROSS-REFERENCE TO RELATED APPLICATIONS

This application claims the priority benefit under 35 U.S.C. §119(e) of U.S. Provisional Application No. 61/173,874, filed on Apr. 29, 2009, the disclosure of which is incorporated herein by reference in its entirety.

### STATEMENT OF GOVERNMENT INTEREST

This invention was made with government support under Grant Number EEC-0647560, awarded by the National Science Foundation (NSF), and Grant Number 5U54 CA119341, awarded by the National Institutes of Health (NIH). The government has certain rights in the invention.

### FIELD OF THE INVENTION

The present invention is directed to compositions and methods of use of a functionalized nanoparticle having a catalytic metal deposit.

### BACKGROUND OF THE INVENTION

Sensitive, rapid and selective immunoassays capable of multiplexed protein detection are critical for clinical applications [Kodadek, Chem. Biol. 8: 105-115 (2001)]. For instance, in many kinds of cancers, following the disease during the course of and after treatment require the detection of multiple protein markers [Ferrari, Nat. Rev. Cancer 5: 161-171 (2005); Sidransky, Nat. Rev. Cancer 2: 210-219 (2002)]. The gold standard for protein detection, the enzyme-linked immunosorbent assay (ELISA), is often not sensitive enough to diagnose some diseases [Barletta et al., Am. J. Clin. Path. 122: 20-27 (2004); Maia et al., J. Virol. Methods 52: 273-286 (1995)]. In addition, multiplexed detection with ELISA has drawbacks such as overlapping spectral features and the need for complex instrumentation for signal readout [MacBeath, Nat. Genet. 32 Suppl: 526-532 (2002)].

Antibody microarrays have emerged as a promising method for multiplexed detection of protein biomarkers [MacBeath, Nat. Genet. 32 Suppl: 526-532 (2002); Angenendt, Drug Discovery Today 10: 503-511 (2005); Ekins, Clin. Chem. 44: 2015-2030 (1998)]. Typically, these microarrays are functionalized with capture antibodies, which bind the protein targets. Next, a second fluorophore-labeled antibody binds the targets forming a sandwich structure detectable with typical DNA microarray detection instrumentation. One limitation of the technique is its sensitivity [Schweitzer et al., Nat. Biotechnol. 20: 359-365 (2002)]. The use of amplification methods, such as immuno-PCR or rolling circle amplification, have been used to enhance sensitivity [Schweitzer et al., Nat. Biotechnol. 20: 359-365 (2002)] but require complicated, multistep protocols [Niemeyer et al., Trends Biotechnol. 23,208-216 (2005)].

Polyvalent polynucleotide gold nanoparticle (Au NP) conjugates [Mirkin et al., Nature 382: 607-609 (1996)] have been utilized as probes for nucleic acids [Elghanian et al., Science 277: 1078-1081 (1997); Storhoff et al., J. Am. Chem. Soc. 120: 1959-1964 (1998); Seferos et al., J. Am. Chem. Soc. 129: 15477-15479 (2007)], proteins [Nam et al., J. Am. Chem. Soc. 2002, 124: 3820-3821 (2002); Nam et al., Science 301: 1884-1886 (2003); Zheng et al., J. Am. Chem. Soc. 130: 9644-9645 (2008)], metal ions [Lee et al., Angew. Chem., Int. Ed. 46: 4093-4096 (2007); Liu et al., J. Am. Chem. Soc. 125: 6642-6643 (2003); Li et al., Angew. Chem., Int. Ed. 2008, 47, 3927-3931 (2008)], and cancerous cells [Medley et al., Anal. Chem. 80: 1067-1072 (2008)]. In addition, these conjugates are both extraordinarily sensitive and selective labels for microarray-based DNA detection assays [Taton et al., J. Am. Chem. Soc. 123: 5164-5165 (2001); Taton et al., Science 289: 1757-1760 (2000); Cao et al., Science 297: 1536-1540 (2002)]. This assay, called the scanometric assay, has since become an FDA-approved detection method and has spurred the development of many related assays [Nam et al., Science 301: 1884-1886 (2003); Xu et al., Anal. Chem. 79: 6650-6654 (2007); Niemeyer et al., Angew. Chem., Int. Ed. 40: 3685-3688 (2001)]. The key to its high sensitivity is the ability to amplify the light scattering of the Au NP probes with electroless metal deposition. In separate but related experiments, immunoblots using antibody Au NP conjugates as probes have shown that gold deposition gives greater signal amplification than silver deposition [Ma et al., Angew. Chem., Int. Ed. 41: 2176-2179 (2002)].

### SUMMARY OF THE INVENTION

Given the aforementioned advances, the multiplexing utility of protein microarrays, the high sensitivity of Au NP conjugate-based detection systems, and the signal amplification of Au NP initiated gold reduction and subsequent deposition are provided herein. The disclosure therefore provides a simple, rapid, and extremely sensitive microarray-based detection method called the scanometric assay that uses the light scattering of functionalized Au NP conjugates and Au NP initiated metal deposition for signal readout. Compositions of the method are also provided.

Accordingly, the present disclosure provides a composition comprising a functionalized nanoparticle, the nanoparticle having a single catalytic metal deposit, the composition having an average diameter of at least about 250 nanometers. In various aspects, the average diameter is from about 250 nanometers to about 5000 nanometers.

In one embodiment, the nanoparticle is comprised of gold. In another embodiment, the nanoparticle is comprised of silver.

In an embodiment, the nanoparticle is catalytically deposited with a metal. In some aspects, the metal is silver. In some aspects, the metal is gold. In some embodiments, the nanoparticle further comprises a second catalytic metal deposition. In yet further embodiments, the nanoparticle further comprises a third catalytic metal deposition.

In another embodiment, the nanoparticle is functionalized with a polynucleotide. In some aspects, the polynucleotide is DNA. In some aspects, the polynucleotide is RNA.

In some embodiments, the polynucleotide further comprises an antibody associated therewith.

The present disclosure also provides compositions wherein the nanoparticle is functionalized with a polypeptide. In some aspects, the polypeptide is an antibody.

In an embodiment of the disclosure, a method is provided for detecting a target molecule comprising the step of contacting a functionalized nanoparticle in association with the target molecule with a metal enhancing solution under conditions that deposit a metal on the nanoparticle to give an average nanoparticle diameter of at least about 250 nanometers, wherein the depositing results in detection of the target molecule. In various aspects, the contacting takes place on a solid support. In some aspects, the contacting takes place in solution.

In one aspect, the disclosure provides a method further comprising contacting the nanoparticle with a sample comprising a first molecule under conditions that allow complex formation between the nanoparticle and the first molecule.

In another aspect, the disclosure provides a method further comprising detecting the complex.

In some embodiments, methods are provided wherein a second molecule is contacted with the first molecule under conditions that allow complex formation prior to the contacting of the nanoparticle with the first molecule. In various aspects, the second molecule is immobilized on a solid support. In some aspects, the solid support is a microarray.

In further aspects, methods are provided wherein the nanoparticle is in a solution.

In some embodiments, the first molecule is a polypeptide. In some embodiments, the second molecule is a polypeptide. In various aspects, the polypeptide is an antibody.

In some embodiments, methods are provided wherein the first molecule is a polynucleotide. In some embodiments, the second molecule is a polynucleotide. In some aspects, the polynucleotide is DNA. In some aspects, the polynucleotide is RNA.

In some embodiments, the present disclosure provides methods wherein the metal enhancing solution is a silver enhancing solution. In some aspects, the metal enhancing solution is a gold enhancing solution.

In various embodiments, the nanoparticle is functionalized with a polynucleotide. In some aspects, the polynucleotide is DNA. In some aspects, the polynucleotide is RNA. In some embodiments, methods are provided further comprising a polypeptide associated therewith. In some aspects, the polypeptide is an antibody.

In some embodiments, methods are provided wherein the nanoparticle is functionalized with a polypeptide. In some aspects, the polypeptide is an antibody.

In some embodiments, the disclosure provides compositions and methods wherein the nanoparticle is comprised of gold.

### BRIEF DESCRIPTION OF THE FIGURES

FIG. 1 depicts scanometric identification (left) and the corresponding quantization (right) of the net signal intensities of various concentrations of PSA in buffer after (a) one silver deposition, (b) two silver depositions, (c) one gold deposition, (d) two gold depositions, and (e) three gold depositions. The light scattering signal was saturated at 65 536 (2¹⁶) units. The gray scale images from the Verigene Reader system were converted into colored ones using GenePix Pro 6 software (Molecular Devices). The exposure time was 500 milliseconds.

FIG. 2 depicts scanometric measurement of PSA concentration in 10% donkey serum and the corresponding quantification of the light scattering signal after two gold depositions. The gray scale images from the Verigene Reader™ system were converted into colored ones using GenePix Pro 6 software (Molecular Devices).

FIG. 3 depicts representative scanning electron microscopy (SEM) images of Au NP probes developed with (a) three silver depositions and (b) three gold depositions.

FIG. 4 depicts scanometric identification of three protein cancer markers for eight different samples in buffer after two gold depositions. The concentration of each antigen was 1.4 pM. (1) All targets present; (2) hCG and PSA; (3) hCG and AFP; (4) PSA and AFP; (5) AFP; (6) PSA; (7) hCG; (8) no targets present. The gray scale images from the Verigene Reader system were converted into colored ones using GenePix Pro 6 software (Molecular Devices), and the exposure time was 200 milliseconds.

FIG. 5 depicts scanometric identification of three cancer markers for eight different samples in 10% donkey serum after two gold depositions. The concentration of each cancer marker was kept constant at 10 pM. a) All targets present; b) hCG and PSA; c) hCG and AFP; d) PSA and AFP; e) AFP; f) PSA; g) hCG; h) Targets not present.

### DETAILED DESCRIPTION OF THE INVENTION

The present disclosure is directed to compositions and their use for detecting a target molecule. In brief, a functionalized nanoparticle in a complex with a target molecule is deposited with a metal which enhances detection of the complex. The compositions and methods provide a simple, rapid, and extremely sensitive detection method that uses light scattering of functionalized Au NP conjugates and Au NP initiated metal deposition for signal readout.

In some aspects, compositions and methods of the present disclosure advantageously improve the signal from any microarray-based detection method, including but not limited to those for DNA [Taton et al., Science 289, 1757-1760 (2000)], metal ions [Lee et al., Anal. Chem. 80, 6805-6808(2008)] and the biobarcode assay [Nam et al., Science 301, 1884-1886 2003)].

In other aspects, the compositions and methods provide improved detection of a target molecule in a solution assay.

A “molecule” as used herein includes a polynucleotide, a polypeptide and a metal ion, each as defined herein.

It is noted here that, as used in this specification and the appended claims, the singular forms “a,” “an,” and “the” include plural reference unless the context clearly dictates otherwise.

It is further noted that the terms “attached,” “conjugated” and “functionalized” are also used interchangeably herein and refer to the association of a polypeptide, a polynucleotide or combinations of a polypeptide and polynucleotide with a nanoparticle.

It is also noted that the term “about” as used herein is understood to mean approximately.

“Hybridization” means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

A “complex” as used herein is a composition with a target molecule in association with a nanoparticle. In various aspects, a complex arises from hybridization of a target polynucleotide target molecule with an polynucleotide functionalized on a nanoparticle, interaction of a polypeptide target molecule with a polypeptide binding molecule functionalized on a nanoparticle, interaction between a target polypeptide with an aptamer functionalized on a nanoparticle, or interaction of a metal ion with a polynucleotide-functionalized nanoparticle.

### Metal Deposition

The present disclosure is directed to compositions and methods comprising a functionalized nanoparticle, the nanoparticle having a single catalytic metal deposit, the composition having an average diameter of at least about 250 nanometers. In various aspects, a composition comprising additional catalytic metal deposits is contemplated. For example and without limitation, a composition comprising 1, 2, 3, 4 or more additional catalytic metal deposits is contemplated by the present disclosure. In some aspects, the metal is gold. In some aspects, the metal is silver. Combinations of gold and silver depositions are also contemplated by the present disclosure. For example and without limitation, where three metal depositions are desired, the composition can comprise one deposition of silver, a second deposition of gold, and a third deposition of silver.

The number of deposits that are added onto a complex will depend on the degree of sensitivity of detection required. The compositions and methods of the present disclosure allow for a “multistage development” in which quantification over a large concentration range is enabled, and additionally yields increased sensitivity. For example and without limitation, the present disclosure provides compositions and methods that enable detection of a target molecule wherein the concentration of the target molecule ranges from about 1 millimolar (mM) to about 100 attomolar (aM). One of ordinary skill in the art will be able to determine the number of rounds of metal deposition for a given application using routine experimentation.

Methods of metal deposition contemplated by the present disclosure include any method known in the art, but specifically exclude methods in which nanoparticles are added to a complex between successive metal depositions. Accordingly, methods according to the present disclosure expressly exclude a step of adding additional nanoparticles between successive metal depositions after a first metal deposition is added to a formed complex.

### Complex Diameter Following Metal Deposition

As described herein, the present disclosure is directed to compositions and methods comprising a functionalized nanoparticle, the nanoparticle having a single catalytic metal deposit, the composition having an average diameter of at least about 250 nanometers. In various aspects, additional catalytic metal deposits are contemplated. For example and without limitation, 1, 2, 3, 4, 5 or more additional catalytic metal deposits are contemplated by the present disclosure. In general, additional catalytic metal deposits correlate with increased detection and increased sensitivity. As described above, however, the skilled artisan can tailor the number of depositions, and resulting average diameter of the complex, according to the desired application.

Accordingly, the average diameter of a complex comprising a composition of the present disclosure is at least about 250 nanometers to about 5000 nanometers. In various aspects, the average diameter of a complex comprising a composition of the present disclosure is about 260, about 270, about 280, about 290, about 300, about 310, about 320, about 330, about 340, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, or about 5000 or more nanometers.

### Nanoparticles

In some embodiments, nanoparticles are provided which are functionalized to have a polynucleotide attached thereto. The size, shape and chemical composition of the nanoparticles contribute to the properties of the resulting polynucleotide-functionalized nanoparticle. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37(1999); Marinakos et al., Chem. Mater. 10: 1214-19(1998); Enustun & Turkevich, J. Am. Chem. Soc. 85: 3317(1963).

Nanoparticles can range in size from about 1 nanometer (nm) to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

### Polynucleotides

Polynucleotides contemplated by the present disclosure include DNA, RNA and modified forms thereof as defined herein. A polynucleotide as disclosed herein is, in some aspects, functionalized on the surface of a nanoparticle. In these aspects, the polynucleotide recognizes and associates with a molecule as defined herein. Accordingly, in some aspects, a polynucleotide is a molecule that is recognized by and associates with a functionalized nanoparticle.

A “polynucleotide” is understood in the art to comprise individually polymerized nucleotide subunits. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleotides include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5 ,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof, generally comprise a polynucleotide from about 5 nucleotides to about 100 nucleotides in length. More specifically, nanoparticles are functionalized with polynucleotides that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length are contemplated.

Polynucleotides, as defined herein, also includes aptamers. The production and use of aptamers is known to those of ordinary skill in the art. In general, aptamers are nucleic acid or peptide binding species capable of tightly binding to and discreetly distinguishing target ligands [Yan et al., RNA Biol. 6(3) 316-320 (2009), incorporated by reference herein in its entirety]. Aptamers, in some embodiments, may be obtained by a technique called the systematic evolution of ligands by exponential enrichment (SELEX) process [Tuerk et al., Science 249:505-10 (1990)]. Aptamers may be comprised of RNA, DNA, or peptide sequences. General discussions of nucleic acid and peptide aptamers are found in, for example and without limitation, Nucleic Acid and Peptide Aptamers: Methods and Protocols (Edited by Mayer, Humana Press, 2009) and Crawford et al., Briefings in Functional Genomics and Proteomics 2(1): 72-79 (2003). In various aspects, an aptamer is between 10-100 nucleotides or amino acids in length.

### Modified Polynucleotides

As discussed above, modified polynucleotides are contemplated for functionalizing nanoparticles. In various aspects, a polynucleotide functionalized on a nanoparticle is completely modified or partially modified. Thus, in various aspects, one or more, or all, sugar and/or one or more or all internucleotide linkages of the nucleotide units in the polynucleotide are replaced with “non-naturally occurring” groups.

In one aspect, this embodiment contemplates a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotide.”

Modified polynucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the polynucleotide consists of 2 to 4, desirably 3, groups/atoms selected from —CH₂—, —O—, —S—, —NRH—, >C═O, >C═NRH, >C═S, —Si(R″)₂—, —SO—, —S(O)₂—, —P(O)₂—, —PO(BH₃)—, —P(O,S)—13 , —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂—, —CH₂—CO—CH₂—, —CH₂—CHOH—CH₂—, —O—CH2-O—, —O—CH2-CH2—, —O—CH2-CH=(including R5 when used as a linkage to a succeeding monomer), —CH₂—CH₂—O—, —NRH—CH₂—CH₂—, —CH₂—CH₂—NRH—, —CH₂—NRH—CH₂—, —O—CH₂—CH₂—NRH—, —NRH—CO—O—, —NRH—CO—NRH—, —NRH—CS—NRH—, —NRH—C(═NRH)—NRH—, —NRH—CO—CH₂—NRH—O—CO—O—, —O—CO—CH₂—O—, —O—CH₂—CO—O—, —CH₂—CO—NRH—, —O—CO—NRH—, —NRH—CO—CH₂—, —O—CH₂—CO—NRH—, —O—CH₂—CH₂—NRH—, —CH═N—O—, —CH₂—NRH—O—, —CH₂—O—N=(including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, —S—CH₂—O—, —CH₂—CH₂—S—, —O—CH₂—CH₂—S—, —S—CH₂—CH=(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHRN)—O—, —O—P(O)₂—NRH H—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—O—P(—O,S)—O—, —O—P(S)₂—O—, —NRH P(O)₂—O—, —O—P(O,NRH)—O—, —O—PO(R″)—O—, —O—PO(CH₃)—O—, and —O—PO(NHRN)—O—, where RH is selected form hydrogen and C1-4-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Application No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH2)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′—CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂-)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

In some aspects, a modified polynucleotide further comprises a polypeptide attached thereto. Thus, a polypeptide in some aspects is associated with the nanoparticle through a polynucleotide. In some aspects, the polypeptide is an antibody, or an antigen binding fragment thereof, but any polypeptide disclosed herein is contemplated for association with a nanoparticle through a polynucleotide.

Methods for associating a polypeptide to a polynucleotide are known to those or ordinary skill in the art and are generally described in Bioconjugate Techniques, 2nd Ed. By Hermanson. Academic Press, London, 2008.

### Polynucleotide Attachment to a Nanoparticle

Polynucleotides contemplated for use in the methods include those bound to the nanoparticle through any means. Regardless of the means by which the polynucleotide is attached to the nanoparticle, attachment in various aspects is effected through a 5′ linkage, a 3′ linkage, some type of internal linkage, or any combination of these attachments.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Accordingly, in some embodiments, the disclosure contemplates that a polynucleotide attached to a nanoparticle is RNA.

In some embodiments, the polynucleotide attached to a nanoparticle is DNA. When DNA is attached to the nanoparticle, the DNA is comprised of a sequence that is sufficiently complementary to a target sequence of a polynucleotide such that hybridization of the DNA polynucleotide attached to a nanoparticle and the target polynucleotide takes place, thereby associating the target polynucleotide to the nanoparticle. The DNA in various aspects is single stranded or double-stranded, as long as the double-stranded molecule also includes a single strand sequence that hybridizes to a single strand sequence of the target polynucleotide. In some aspects, hybridization of the polynucleotide functionalized on the nanoparticle can form a triplex structure with a double-stranded target polynucleotide. In another aspect, a triplex structure can be formed by hybridization of a double-stranded polynucleotide functionalized on a nanoparticle to a single-stranded target polynucleotide.

### Polypeptides

As used herein a “polypeptide” refers to a polymer comprised of amino acid residues. In some aspects of the disclosure, a polypeptide is functionalized to a nanoparticle as described below. In related aspects, the polynucleotide-functionalized nanoparticle recognizes and associates with a target molecule and enables detection of the target molecule.

In some embodiments, a polypeptide is a molecule that is recognized and detected as a result of its association with a functionalized nanoparticle as described herein.

Polypeptides of the present disclosure may be either naturally occurring or non-naturally occurring.

### Naturally Occurring Polypeptides

Naturally occurring polypeptides include without limitation biologically active polypeptides and antibodies that exist in nature or can be produced in a form that is found in nature by, for example, chemical synthesis or recombinant expression techniques. Naturally occurring polypeptides also include lipoproteins and post-translationally modified proteins, such as, for example and without limitation, glycosylated proteins.

Antibodies contemplated for use in the methods and compositions of the present disclosure include without limitation antibodies that recognize and associate with cancer markers, cardiac markers (for example and without limitation, troponin), and viral markers (for example and without limitation, HIV p24).

### Non-Naturally Occurring Polypeptides

Non-naturally occurring polypeptides contemplated by the present disclosure include but are not limited to synthetic polypeptides, as well as fragments, analogs and variants of naturally occurring or non-naturally occurring polypeptides as defined herein. Non-naturally occurring polypeptides also include proteins or protein substances that have D-amino acids, modified, derivatized, or non-naturally occurring amino acids in the D- or L- configuration and/or peptidomimetic units as part of their structure. The term “protein” typically refers to large polypeptides. The term “peptide” typically refers to short polypeptides.

Non-naturally occurring polypeptides are prepared, for example, using an automated polypeptide synthesizer or, alternatively, using recombinant expression techniques using a modified polynucleotide which encodes the desired polypeptide.

As used herein a “fragment” of a polypeptide is meant to refer to any portion of a polypeptide or protein smaller than the full-length polypeptide or protein expression product.

As used herein an “analog” refers to any of two or more polypeptides substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it.

As used herein a “variant” refers to a polypeptide, protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art. In various aspects, polypeptides are modified by glycosylation, pegylation, and/or polysialylation.

Fusion proteins, including fusion proteins wherein one fusion component is a fragment or a mimetic, are also contemplated. This group also includes antibodies along with fragments and derivatives thereof, including but not limited to Fab′ fragments, F(ab)₂ fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

### Polypeptide Attachment to a Nanoparticle

In some embodiments, a polypeptide is attached to a nanoparticle. In one aspect, a polypeptide is directly associated with the nanoparticle. In another aspect, the polypeptide is associated with the nanoparticle indirectly. In further aspects, the indirect association is effected by way of a polypeptide being attached to a polypeptide, which is itself directly associated with the nanoparticle. In another aspect, the polypeptide is indirectly associated with the nanoparticle through its associations with a spacer as defined herein. Any means of associating a polypeptide with a nanoparticle are contemplated by the present disclosure and are understood by those of ordinary skill in the art [see Bioconjugate Techniques, 2nd Ed. By Hermanson. Academic Press, London, 2008].

### Target Molecules

In some embodiments, the present disclosure is directed to contacting a target molecule with a functionalized nanoparticle to form a complex, and further comprising depositing a metal on the complex to enable its detection. In various aspects, the target molecule is a polypeptide as defined herein.

In various aspects, target polypeptides contemplated by the present disclosure include but are not limited to cancer antigen 150 (CA150), Cancer antigen (CA19), cancer antigen (CA50), calcium binding protein 39-like (CAB39L), CD22, CD24, CD5, CD19, CD63, CD66, Carcinoembryonic antigen-related cell adhesion molecule 1 (biliary glycoprotein) (CEACAM1), carcinoembryonic antigen-related cell adhesion molecule 5 (CEACAM5), clusterin associated protein 1 (CLUAP1), cancer/testis antigen 1B (CTAG1B), cancer/testis antigen 2 (CTAG2), cutaneous T-cell lymphoma-associated antigen 5 (CTAGE5), carcinoembryonic antigen (CEA), estrogen receptor-binding fragment-associated antigen 9 (EBAG9), FAM120C, FLJ14868, formin-like protein 1 (FMNL1), G antigen 1 (GAGE1), glycoprotein A33 (transmembrane) (GPA33), ganglioside OAcGD3, heparanase 1, Jak and microtubule interacting protein 2 (JAKMIP2), leucine-rich repeats and immunoglobulin-like domains 3 (LRIG3), leucine rich repeat containing 15 (LRRC15), lung carcinoma Cluster 2, melanoma-associated antigen 1 (MAGE 1), melanoma antigen family A, 10 (MAGEA10), melanoma antigen family A, 11 (MAGEA11), melanoma antigen family A, 12 (MAGEA12), melanoma antigen family A, 2 (MAGEA2), melanoma antigen family A, 4 (MAGEA4), melanoma antigen family B, 1 (MAGEB1), melanoma antigen family B, 2 (MAGEB2), melanoma antigen family B, 3 (MAGEB3), melanoma antigen family B, 4 (MAGEB4), melanoma antigen family B, 6 (MAGEB6), melanoma antigen family C, 1 (MAGEC1), melanoma antigen family E, 1 (MAGEE1), melanoma antigen family H, 1 (MAGEH1), melanoma antigen family L 2 (MAGEL2), meningioma expressed antigen 5 (hyaluronidase), (MGEA5), MOK protein kinase, mucin 16, cell surface associated (MUC16), mucin 4, cell surface associated (MUC4), melanoma associated antigen, mesothelin, mucin 5AC, nestin, ovarian cancer immuno-reactive antigen domain containing 1 (OCIAD1), opa interacting protein 5 (OIP5), ovarian carcinoma-associated antigen, PAGE4, proliferating cell nuclear antigen (PCNA), preferentially expressed antigen in melanoma (PRAME), prostate tumor overexpressed 1 (PTOV1), plastin L, prostate cell surface antigen, prostate mucin antigen/PMA, RAGE, RASD2, ring finger protein 43 (RNF43), ropporin, rhophilin associated protein 1 (ROPN1), ribosomal protein, large, P2 (RPLP2), squamous cell carcinoma antigen recognized by T cell 2 (SART2), squamous cell carcinoma antigen recognized by T cells 3 (SART3), small breast epithelial mucin (SBEM), serologically defined colon cancer antigen 10 (SDCCAG10), serologically defined colon cancer antigen 8 (SDCCAG8), sel-1 suppressor of lin-12-like (C. elegans) (SEL1L), human sperm protein associated with the nucleus on the X chromosome (SPANX), SPANXB1, synovial sarcoma, X breakpoint 5 (SSX5), six-transmembrane epithelial antigen of prostate 4 (STEAP4), serine/threonine kinase 31 (STK31), tumor associated glycoprotein (TAG72), tumor endothelial marker 1 (TEM1), X antigen family, member 2 (XAGE2). Additional target polypeptides contemplated by the present disclosure include without limitation cardiac markers (for example and without limitation, troponin), viral markers (for example and without limitation, HIV p24).

In some aspects, the target molecule is a polynucleotide as defined herein. Any target polynucleotide is contemplated for use with the methods of the present disclosure, including but not limited to the polynucleotides encoding the target polypeptides disclosed herein. Of course, the skilled artisan can easily design a polynucleotide sequence that associates with any desired target polynucleotide. The present disclosure is therefore not limited in scope by the target molecules disclosed herein.

In further embodiments the target molecule is an ion. The present disclosure contemplates that in one aspect the ion is nitrite (NO₂⁻). In some aspects, the ion is a metal ion that is selected from the group consisting of mercury (Hg²⁺), Cu²⁺ and UO²⁺.

### Methods

Methods described herein are directed to depositing a metal on a complex formed between a functionalized nanoparticle and a target molecule to enhance detection of the complex. Metal is deposited on the nanoparticle/target molecule when the nanoparticle/target molecule complex is contacted with a metal enhancing solution under conditions that cause a layer of the metal to deposit on the complex.

A metal enhancing solution, as used herein, is a solution that is contacted with a functionalized nanoparticle-target molecule complex to deposit a metal on the complex. In various aspects and depending on the type of metal being deposited, the metal enhancing solution comprises, for example and without limitation, HAuCl₄, silver nitrate, NH₂OH and hydroquinone.

In some embodiments, the target molecule is immobilized on a support when it is contacted with the functionalized nanoparticle. A support, as used herein, includes but is not limited to a column, a membrane, or a glass or plastic surface. A glass surface support includes but is not limited to a bead or a slide. Plastic surfaces contemplated by the present disclosure include but are not limited to slides, and microtiter plates. Microarrays are additional supports contemplated by the present disclosure, and are typically either glass, silicon-based or a polymer. Microarrays are known to those of ordinary skill in the art and comprise target molecules arranged on the support in addressable locations. Microarrays can be purchased from, for example and without limitation, Affymetrix, Inc.

In some embodiments, the target molecule is in a solution. In this type of assay, a functionalized nanoparticle is contacted with the target molecule in a solution to form a nanoparticle/target molecule complex that is then detected following deposition of a metal on the complex. Methods of this type are useful whether the target molecule is in a solution or in a body fluid. For example and without limitation, a solution as used herein means a buffered solution, water, or an organic solution. Body fluids include without limitation blood (serum or plasma), lymphatic fluid, cerebrospinal fluid, semen, urine, synovial fluid, tears, mucous, and saliva and can be obtained by methods routine to those skilled in the art.

The disclosure also contemplates the use of the compositions and methods described herein for detecting a metal ion (for example and without limitation, mercuric ion (Hg²⁺)). In these aspects, the method takes advantage of the cooperative binding and catalytic properties of DNA-functionalized nanoparticles and the selective binding of a thymine-thymine mismatch for Hg²⁺ [Lee et al., Anal. Chem. 80: 6805-6808 (2008)].

Methods described herein are also contemplated for use in combination with the biobarcode assay. The biobarcode assay is generally described in U.S. Pat. Nos. 6,974,669 and 7,323,309, each of which is incorporated herein by reference in its entirety.

Methods of the disclosure include those wherein silver or gold or combinations thereof are deposited on a functionalized nanoparticle in a complex with a target molecule.

In one embodiment, methods of silver deposition on a functionalized nanoparticle complex as described herein yield a limit of detection of a target molecule of about 3 pM after a single silver deposition. In another aspect, a second silver deposition improves the limit of detection to about 30 fM. Thus, the number of depositions of silver relates to the limit of detection of a target molecule. Accordingly, one of ordinary skill in the art will understand that the methods of the present disclosure may be tailored to correlate with a given concentration of target molecule. For example and without limitation, for a target molecule concentration of 30 fM, two silver depositions can be used. Concentrations of target molecule suitable for detection by silver deposition are about 3 pM, about 2 pM, about 1 pM, about 0.5 pM, about 400 fM, about 300 fM, about 200 fM, about 100 fM or less.

The amount of time that the functionalized nanoparticle complex is exposed to a metal enhancing solution is about 5 minutes. The amount of time that the functionalized nanoparticle complex is exposed to a metal enhancing solution is about 1, about 2, about 3, about 4, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 1 hour, about 2 hours or longer.

The temperature at which the metal deposition takes place is about 0° C. The methods of the present disclosure contemplate a temperature for metal deposition that is about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., or higher.

In another embodiment, methods of gold deposition on a functionalized nanoparticle complex as described herein yield a limit of detection of a target molecule of about 3 pM after one gold deposition. In various aspects, the limit of detection of a target molecule is about 2.5 pM, about 2 pM, about 1.5 pM, about 1 pM, about 0.5 pM, about 400 fM, about 300 fM, about 200 fM, about 100 fM, about 50 fM, about 40 fM, about 30 fM or less after one gold deposition.

In another embodiment, methods of gold deposition on a functionalized nanoparticle complex as described herein have been found to yield a limit of detection of a target molecule of about 300 fM after two gold depositions. In various aspects, the limit of detection of a target molecule is about 250 fM, about 200 fM, about 150 fM, about 100 fM, about 50 fM, about 10 fM, about 5 fM, about 1 fM, about 0.5 aM, about 400 aM, about 300 aM, about 200 aM, about 100 aM or less after two gold depositions.

In methods provided, a functionalized nanoparticle is contacted with a sample comprising a first molecule under conditions that allow complex formation between the nanoparticle and the first molecule. The complex is then detected. Detection can be performed by any means known in the art, and includes but is not limited to visualization by the naked eye and an automated reader system (for example but not limited to a Verigene Reader system).

Method are also provided wherein a second molecule is contacted with the first molecule under conditions that allow complex formation prior to the contacting of the nanoparticle with the first molecule.

Method are also contemplated wherein a target molecule is attached to a second functionalized nanoparticle that associates with the first functionalized nanoparticle. In some aspects, the second functionalized nanoparticle is immobilized on a solid support. In other aspects, the second functionalized nanoparticle is in a solution.

Methods provided generally contemplate use of a composition comprising a functionalized nanoparticle as described herein.

Methods provided also generally contemplate contacting a composition comprising a nanoparticle with more than one target molecule. Accordingly, in some aspects it is contemplated that a nanoparticle which is functionalized with more than one polypeptide and/or polynucleotide, is able to simultaneously recognize and associate with more than one target molecule.

In further embodiments, a target polynucleotide is identified using a “sandwich” protocol for high-throughput detection and identification. For example and without limitation, a polynucleotide that recognizes and selectively associates with the target polynucleotide is immobilized on a solid support. The sample comprising the target polynucleotide is contacted with the solid support comprising the polynucleotide, thus allowing an association to occur. Following removal of non-specific interactions, a composition comprising a functionalized nanoparticle as described herein is added. In these aspects, the nanoparticle is functionalized with a molecule that selectively associates with the target polynucleotide, thus generating the “sandwich” of polynucleotide-target polynucleotide-functionalized nanoparticle. This complex is then exposed to a metal deposition process as described herein, resulting in highly sensitive detection. Quantification of the interaction allows for determinations relating but not limited to disease progression, therapeutic effectiveness, disease identification, and disease susceptibility.

### Spacers

In certain aspects, functionalized nanoparticles are contemplated which include those wherein a polynucleotide is attached to the nanoparticle through a spacer. “Spacer” as used herein means a moiety that does not participate in modulating gene expression per se but which serves to increase distance between the nanoparticle and the polynucleotide, or to increase distance between individual polynucleotides when attached to the nanoparticle in multiple copies, or to increase distance between the therapeutic agent and the nanoparticle. Thus, spacers are contemplated being located between individual polynucleotides in tandem, whether the polynucleotides have the same sequence or have different sequences. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

In certain aspects, the polynucleotide has a spacer through which it is covalently bound to the nanoparticles. These polynucleotides are the same polynucleotides as described above. In instances wherein the spacer is a polynucleotide, the length of the spacer in various embodiments is at least about 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, at least 50 nucleotides, or even greater than 50 nucleotides. The spacer may have any sequence which does not interfere with the ability of the polynucleotides to become bound to the nanoparticles. The spacers should not have sequences complementary to each other or to that of the polynucleotides. In certain aspects, the bases of the polynucleotide spacer are all adenines, all thymines, all cytidines, all guanines, all uracils, or all some other modified base.

### Surface Density

The density of polynucleotides on the surface of the NP can be tuned for a given application. For instance, work by Seferos et al. [Nano Lett., 9(1): 308-311, 2009] demonstrated that the density of DNA on the NP surface affected the rate at which it was degraded by nucleases. This density modification is used, for example, in a NP based therapeutic agent delivery system where a drug and ON-NP enter cells, and the ON is degraded at a controlled rate.

Accordingly, nanoparticles as provided herein have a packing density of the polynucleotides on the surface of the nanoparticle that is, in various aspects, sufficient to result in cooperative behavior between nanoparticles and between polynucleotide strands on a single nanoparticle. In another aspect, the cooperative behavior between the nanoparticles increases the resistance of the polynucleotide to nuclease degradation. In yet another aspect, the uptake of nanoparticles by a cell is influenced by the density of polynucleotides associated with the nanoparticle. As described in PCT/US2008/65366, incorporated herein by reference in its entirety, a higher density of polynucleotides on the surface of a nanoparticle is associated with an increased uptake of nanoparticles by a cell.

A surface density adequate to make the nanoparticles stable and the conditions necessary to obtain it for a desired combination of nanoparticles and polynucleotides can be determined empirically. Generally, a surface density of at least 2 pmoles/cm² will be adequate to provide stable nanoparticle-polynucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

The invention will be more fully understood by reference to the following examples which detail exemplary embodiments of the invention. They should not, however, be construed as limiting the scope of the invention. All citations throughout the disclosure are hereby expressly incorporated by reference.

### Examples

### Example 1

In this example, a microarray sandwich assay was performed for prostate specific antigen (PSA), Scheme 1 (below). PSA was chosen as an initial target molecule because of its importance as a prostate cancer marker [Lilja et al., Nat. Rev. Cancer 8: 268-278 (2008)], and since many assays have been developed for this target molecule [Nam et al., Science 301: 1884-1886 (2003); Oh et al., Small 2: 103-108 (2006); Schweitzer et al., Proc. Natl. Acad. Sci. U.S.A. 97: 10113-10119 (2000); Yu et al., J. Am. Chem. Soc. 128: 11199-11205 (2006); He et al., J. Am. Chem. Soc. 122: 9071-9077 (2000); Goluch et al., Lab Chip 6: 1293-1299 (2006)], there was a good basis for comparison. In a typical experiment, an antibody microarray was fabricated by spotting monoclonal capture antibodies to the surface of N-hydroxysuccinimide-activated glass slides (CodeLink, SurModics). Six spots, all with antibodies for PSA, were used in each assay well. The use of six spots allow one to obtain statistically significant data in each assay. The slides were then passivated with ethanolamine. Probes were prepared by first modifying 13 nm diameter Au NPs with 3′-propylthiol and 5′-decanoic acid modified polynucleotides and then covalently immobilizing antibodies for PSA via carbodiimide coupling [Hermanson, Bioconjugate Techniques; Academic Press: San Diego, Calif., 1996].

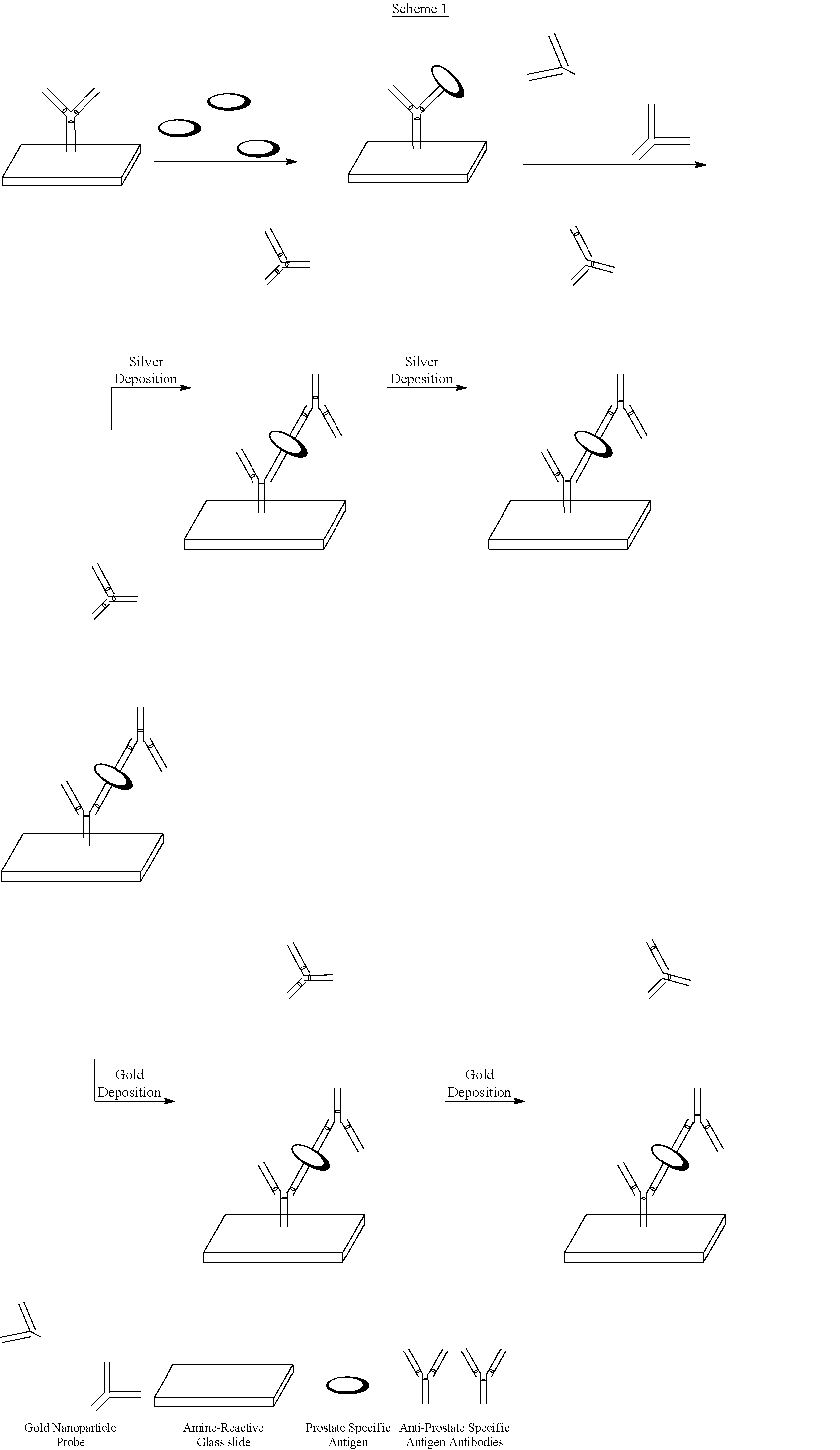

### Preparation of Functionalized Nanoparticles

Thirteen ±1 nm Au NPs were synthesized by the Frens method [Frens, Nature-Phys. Sci. 241, 20-22 (1973)], resulting in approximately 10 nM solutions. The 3′-propylthiol-T₂₄-decanoic acid polynucleotide was synthesized with standard phosphoramidite chemistry reagents purchased from Glen Research and purified with ion exchange HPLC. The polynucleotide Au NP conjugates were synthesized by incubating 3 μM of the polynucleotide with the as-synthesized Au NPs. The conjugates were salted using literature procedures [Hurst et al., Anal. Chem. 78, 8313-8318 (2006)] to a final concentration of 1.0 M NaCl and purified via repeated centrifugation and resuspension in 0.01% Tween 20 in water. The antibodies were conjugated to the polynucleotide modified Au NPs with 1-ethyl-3-(3-dimethylaminopropyl)carbodiimide (EDC) and sulfo N-hydroxysuccinimide (NHS). In this procedure, 10 μL of 0.01% Tween 20 solutions containing 0.5 pmol of the particles were prepared. Five μL of a 30 mM sulfo-NHS solution in a 0.1 M 2-(Nmorpholino) ethanesulfonic acid (MES) buffer at pH 5, followed by 5 μL of 15 mM EDC solution in 0.1 M MES were added to these particles. This mixture was agitated for 15 minutes, and then the particles were purified from excess reagent via centrifugation and resuspension three times in 5 mM MES buffer supplemented with 0.01% Tween 20. After the final centrifugation and supernatant removal, the particles were isolated in 10 μL of oily suspension. To this solution, 5 μg of antibodies in 10 mM phosphate buffer (PB) were added from a 1 mg/mL solution. Last, 5 μL of 0.1 M phosphate buffer (PB), pH 7.4, were added to the mixture, and the solution was agitated overnight at room temperature. The conjugates were purified by repeated centrifugation and resuspension in Dulbecco's PBS with 0.025% Tween 20 and 0.1% BSA. Finally, BSA was added to a final concentration of 1%, and the conjugates were passivated overnight.

### Microarray Preparation

An arrayer equipped with 125 μm diameter pins (GMS 417, Affymetrix) was used for the preparation of the antibody microarrays. The microarrays were fabricated by spotting 250 μg/mL solutions of the antibodies in 0.1M phosphate buffer (PB), pH 8.0, with 150 mM NaCl and 0.001% Tween 20 on to the surface of NHS ester-activated Codelink slides (SurModics Inc.). Six replicate spots for single target molecule detection or three replicate spots of each antibody for multiplexed detection were arrayed at defined locations. The slides were then incubated overnight at 4° C. under an N₂ atmosphere. They were then passivated by incubating them with a 0.2% (v/v) solution of ethanolamine (411000, Aldrich) in 50 mM borate buffer, pH=9.5 overnight at 4° C. Finally, they were then washed with Nanopure water (>18 MΩ, Barnstead International) and spin-dried for one minute.

The microarray of the oligonucleotide-modified Au NP conjugates for SEM imaging was prepared by spotting approximately 400 conjugates to the surface of glass slides (Codelink, SurModics) [Andreeva et al., Colloids Surf., A 300, 300-306 (2007)]. Three replicate spots were arrayed at defined locations. The glass slides were dried, and the diameters of Au NP probes were increased with silver or gold staining solution, gently washed with Nanopure water, and spin-dried. The slides were sputtered with 20 nm of Au/Pd before imaging. All scanning electron microscopy (SEM) images were obtained using a LEO Gemini SEM.

### Example 2

The assay began by incubating the test solution with PSA at a designated concentration for 1 hour at room temperature on the chip with capture antibodies (assay buffer: Dulbecco's PBS with 0.1% Tween 20, 0.1% BSA, and 1% poly(acrylic acid)). The assay buffer was prepared by adding 500 μL of a 10% bovine serum albumin (BSA) solution (DY995, R&D Systems), 500 μL of an aqueous 10% Tween 20 solution (Sigma), and 500 mg of poly(acrylic acid) (420344, Sigma) to Dulbecco's phosphate-buffered saline (PBS) buffer (Invitrogen) in a final volume of 50 mL.

### Antibodies and Antigens

The proteins used in the study were prostate specific antigen (PSA) (P3338, Sigma-Aldrich), the spotted PSA antibody (ab403, Abcam), the Au NP PSA antibody (AF1344, R&D Systems), R-fetoprotein antigen (APF) (A32260H, Biodesign International), the spotted AFP antibody (10-A05, clone M19301, Fitzgerald Industries International, Inc.), the Au NP AFP antibody (70-XG05, Fitzgerald Industries International, Inc.), human chorionic gonadotropin (HCG) (A81355M, Biodesign International), the spotted HCG antibody (E20579, Biodesign International), and the Au NP-monoclonal HCG antibody (E20106, Biodesign International).

### Example 3

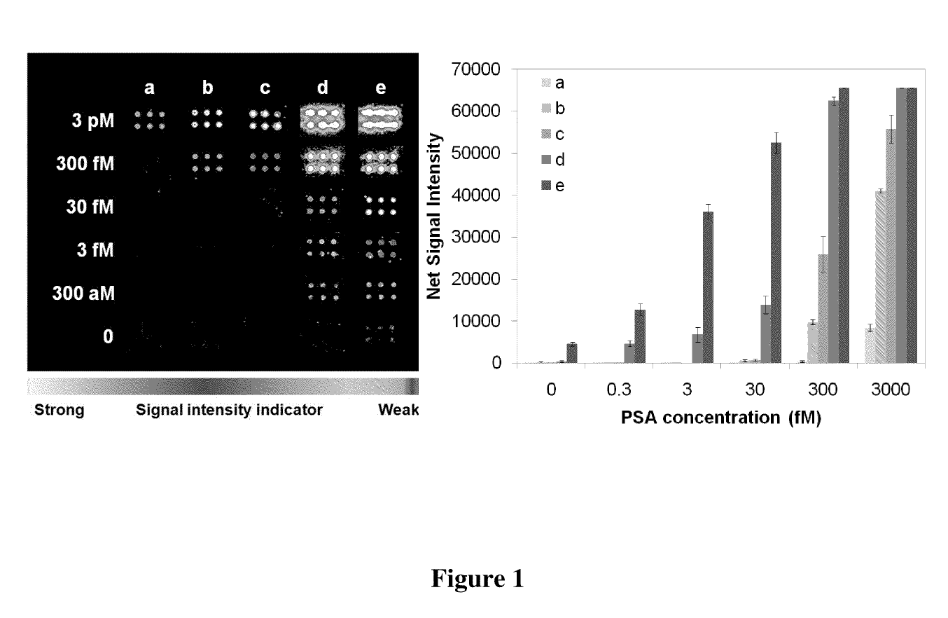

Since each chip had ten different wells (in addition to six capture spots in each well), multiple separate assays can be carried out at once (top to bottom, FIG. 1). After washing the slide with assay buffer, 150 pM of the Au NP probes in assay buffer were incubated with the microarray-bound targets for 1 hour at room temperature. The slides were washed again. To increase the light scattering signal of the immobilized Au NP probes, gold or silver was catalytically deposited on them using electroless deposition techniques (left to right, FIG. 1). Gold(III) chloride trihydrate (520918, Aldrich) and hydroxylamine hydrochloride (159417, Aldrich) were used for preparing gold enhancing solution. Normal donkey serum (Chemicon International, Temecula, Calif.) was used as received.

The antibody microarray was assembled with a 10-well manual hybridization chamber. Antibody spots on the microarray were arrayed at defined locations across the glass slides so that multiple tests could be performed on the single slide by isolating reaction sites with silicone gaskets to create individual wells. Each well of the chamber was filled with 50 μL of antigen solution and allowed to incubate for 1 hour at room temperature with shaking at 1200 rpm. After washing the chambers three times with assay buffer, 50 μL of 150 pM Au NP probes in assay buffer were incubated with the slides for 1 hour at room temperature. The concentration of each of the Au NP probes was 150 pM in multiplexed detection experiments. The chamber was again washed three times and then disassembled. The slide was rinsed with Dulbecco's PBS with 0.1% Tween 20 and Nanopure water and spin-dried for one minute. The slide was then developed with silver or gold enhancing solution (1:1 (v:v) mixture of 5 mM HAuCl₄ and 10 mM NH₂OH) for 5 min and imaged with a Verigene Reader system. The light scattering was quantified with the Verigene Reader system, which is a device that captures evanescent wave-induced light scattering from the amplified Au NPs. The Verigene Reader light scattering reader system, silver enhancing solutions, and 10-well manual hybridization chambers were purchased from Nanosphere, Inc.

In a conventional scanometric detection experiment, electroless silver deposition is used to grow Au NP probes on oligonucleotide microarrays [Taton et al., Science 289: 1757-1760 (2000)], FIG. 1a. When PSA was used as the target molecule under the conditions described above, the limit of detection (LOD) is 3 pM when silver was the amplifying agent. Interestingly, the silverplated Au NP conjugates could be used as nucleation agents to perform a second silver deposition on the same microarray, which improves the LOD to 30 fM, FIG. 1b. Others have shown that a second round of silver development increases the limit of detection of immunoblots [Ma et al., Angew. Chem., Int. Ed. 41: 2176-2179 (2002), and immunosorbent assays [Shim et al., Nanomedicine 3: 485-493 (2008)]. The increase in signal arises from particle growth (vide infra), because on the nano- and microscale light scattering intensity increases dramatically with particle diameter [Jain et al., J. Phys. Chem. B 110,7238-7248 (2006)]. A third round of silver deposition did not significantly improve the assay LOD due to increased background signal, but is contemplated for use under conditions of low target molecule concentration.

Methods of electroless deposition using HAuCl₄ and NH₂OH have been used to increase the diameter of Au NPs in solution [Brown et al., Langmuir 14: 726-728 (1998)], and in immunoblots [Ma et al., Angew. Chem., Int. Ed. 41: 2176-2179 (2002)]. A microarray developed with these reagents resulted in an LOD of 30 fM, comparable to that of two sequential silver depositions, FIG. 1c. An additional treatment with the gold development solution improved the LOD to 300 aM, FIG. 1d. A third deposition of gold increased the light scattering signal but did not improve the LOD due to increased background signal, FIG. 1e.

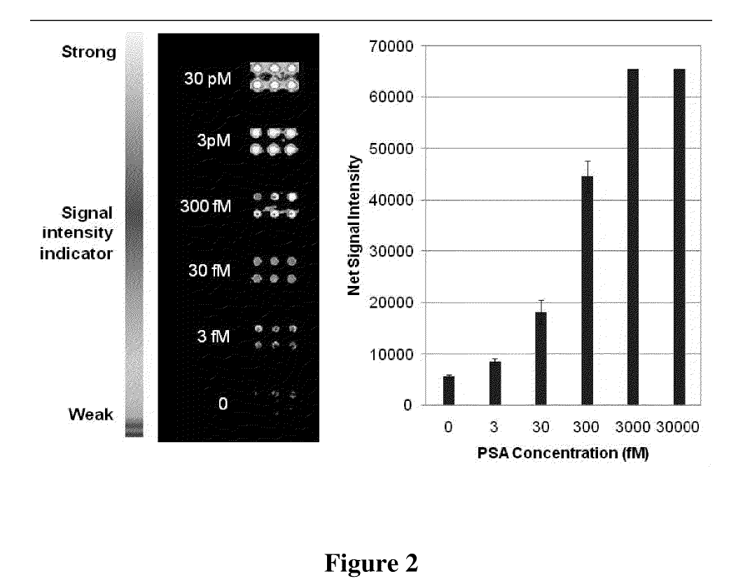

As one moves to more complex matrixes, assay LODs are often challenged due to increased background. When this assay was carried out in 10% serum, the LOD was 3 fM with two gold depositions, FIG. 2. This LOD is approximately 3 orders of magnitude lower than that of commercially available ELISA assays for PSA (approximately picomolar concentration) [Ward et al., Ann. Clin. Biochem 38: 633-651 (2001)].

One of the unique features of a multistage development is that it allows for quantification over a large concentration range in addition to increased sensitivity. With one gold deposition, the dynamic range of this assay in buffer is between 30 fM and 3 pM, and with two, it is between 300 aM and 300 fM, FIG. 1. Therefore, with two serial gold depositions, this scanometric assay is capable of PSA detection over a 4 order of magnitude concentration range.

### Example 4

To better understand the reason why multiple gold depositions provide better signal than one silver deposition, the growth of Au NP probes were investigated by scanning electron microscopy (SEM) after various metal deposition procedures. In a typical experiment, a microarrayer was used to deposit approximately 400 Au NPs per spot on glass slides [Andreeva et all., Colloids Surf., A 300, 300-306 (2007)], and then the size of the Au NP probes were measured after silver or gold development. With silver, the average diameters of the probes were 100±25, 270±130, and 550±140 nm after one, two, and three developments, respectively. With gold, the developed probe diameters are 420±100, 1400±470, and 2700±710 nm after one, two, and three depositions, respectively. These data indicate that repeated metal depositions increase the average probe diameter. Larger nano- and microstructures scatter light better than smaller ones [Jain et al., J. Phys. Chem. B 110: 7238-7248 (2006); Yguerabide et al., Anal. Biochem. 262: 157-176 (1998); Yguerabide et al., Anal. Biochem. 262: 137-156 (1998)] which correlates with increased light scattering intensity as seen in FIG. 1.

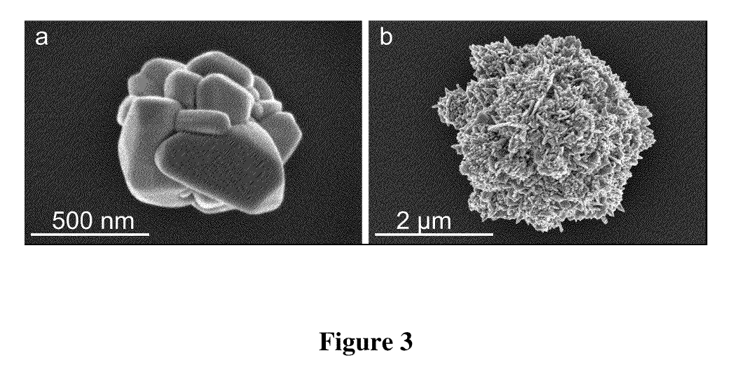

The greater signal amplification observed when gold deposition is used versus silver deposition likely arises from their different growth mechanisms. Typically, silver deposition causes the autocatalytic reduction of silver on the Au NPs [Taton et al., Science 289: 1757-1760 (2000)], increasing the size of the structure, which results in signal enhancement, FIG. 3a. The gold development solution, however, likely leads to the continuous nucleation of new Au NPs by the probe Au NPs in addition to autocatalytic growth. These newly nucleated particles aggregate on the probe Au NPs, resulting in signal enhancement and gold microstructures that are larger than those developed by silver, FIG. 3b. The nucleation of new particles by existing Au NPs has been observed in the seed-mediated synthesis of Au NPs [Jana et al., J. Chem. Mater. 13, 2313-2322 (2001)].

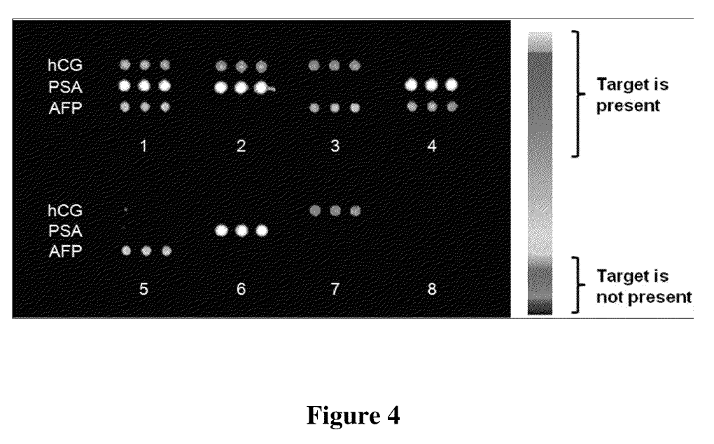

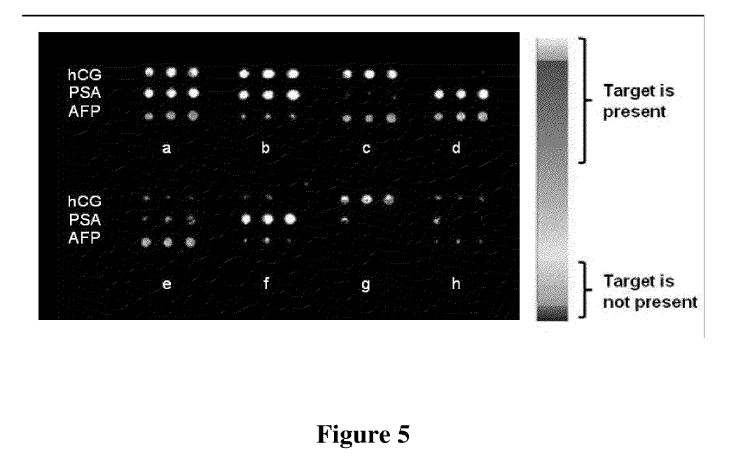

After the origin of the increased signal using gold development was determined, the scanometric immunoassay was challenged with detecting three protein cancer markers using multiple gold depositions. Multiplexed protein analysis is becoming increasingly important for disease diagnosis, and high selectivity is critical for the success of multiplexing assays [Ferrari, Nat. Rev. Cancer 5, 161-171 (2005)]. In a typical experiment, antibodies to PSA, human chorionic gonadotropin (hCG), a testicular cancer marker, and R-fetoprotein (AFP), a hepatic cancer marker, were spotted onto a microarray chip. Next, the target antigens were incubated in the wells. After washing, antibody modified oligonucleotide Au NPs specific for PSA, hCG, or AFP were used to sandwich the antigens. The selectivity of the system was tested by detecting eight different combinations of antigens. In the first well, all three antigens were present. In the next seven wells, different combinations of targets were mixed. The concentrations of each of the target antigens were kept constant at 1.4 pM. After two serial gold depositions the presence of the target in each combination was clearly indicated by the high intensity signal, FIG. 4. In the absence of the protein cancer marker, little signal was observed. This indicates that the assay is capable of highly selective antigen detection. The differences in spot intensity for the different antigens are likely a result of differences in the binding affinity of the antibodies [Stoeva et al., J. Am. Chem. Soc. 128, 8378-8379 (2006)]. Finally, the assay demonstrated high selectivity in 10% serum, FIG. 5.

In conclusion, described herein are methods of multiple gold depositions as a signal enhancing mechanism in a simple, rapid, and ultrahigh sensitivity scanometric assay based on antibody microarrays and Au NP probes. Multiple gold depositions are an alternative light scattering amplification tool for scanometric assays that provide greater signal than the typical single silver deposition. This greater signal arises because the developed probe diameters are much larger and, thus, scatter light better than probes developed by one silver deposition. Gold-developed structures are likely larger than silver developed structures due to the unique growth mechanism of gold deposition. Of course, it will be appreciated that depending on the application, either silver or gold metal deposition is useful in the practice of the methods of the present disclosure.

It will be understood that although the disclosure exemplified the detection of protein cancer markers, the use of multiple gold developments will improve the signal from any high-throughput assay, including those for DNA [Taton et al., Science 289, 1757-1760 (2000)], metal ions [Lee et al., Anal. Chem. 80, 6805-6808(2008)] and the biobarcode assay [Nam et al., Science 301, 1884-1886 2003)]. Ultimately, metal-based signal enhancement could have significant utility in detection schemes as well as in broader clinical and research applications.

1. A composition comprising a functionalized nanoparticle, the nanoparticle having a single catalytic metal deposit, the composition having an average diameter of at least about 250 nanometers.

2. The composition of claim 1 wherein the average diameter is from about 250 nanometers to about 5000 nanometers.

3. The composition of claim 1 wherein the nanoparticle is comprised of gold.

4. (canceled)

5. The composition of claim 1 wherein the metal is silver or gold.

6. The composition of claim 1 further comprising a second catalytic metal deposition.

7. The composition of claim 6 further comprising a third catalytic metal deposition.

8. The composition of claim 1 wherein the nanoparticle is functionalized with a polynucleotide.

9. (canceled)

10. (canceled)

11. The composition of claim 8, the polynucleotide further comprising an antibody associated therewith.

12. The composition of claim 1 wherein the nanoparticle is functionalized with a polypeptide.

13. (canceled)

14. A method for detecting a target molecule comprising the step of contacting a functionalized nanoparticle in association with the target molecule with a metal enhancing solution under conditions that deposit the metal on the nanoparticle to give an average nanoparticle diameter of at least about 250 nanometers, wherein the depositing results in detection of the target molecule.

15. The method of claim 14 wherein the contacting takes place on a solid support or in solution.

16. (canceled)

17. The method of claim 14 further comprising contacting the nanoparticle with a sample comprising a first molecule under conditions that allow complex formation between the nanoparticle and the first molecule.

18. The method of claim 17 further comprising detecting the complex.

19. The method of claim 14 wherein a second molecule is contacted with the first molecule under conditions that allow complex formation prior to the contacting of the nanoparticle with the first molecule.

20. The method of claim 19 wherein the second molecule is immobilized on a solid support.

21. (canceled)

22. The method of claim 17 wherein the first molecule or the second molecule is a polypeptide.

23. (canceled)

24. (canceled)

25. The method of claim 17 wherein the first molecule or the second molecule is a polynucleotide.

26. (canceled)

27. (canceled)

28. (canceled)

29. The method of claim 14 wherein the metal enhancing solution is a silver enhancing solution or a gold enhancing solution.

30. (canceled)

31. The method of claim 14 wherein the nanoparticle is functionalized with a polynucleotide.

32. (canceled)

33. (canceled)

34. (canceled)

35. (canceled)

36. The method of claim 14 wherein the nanoparticle is functionalized with a polypeptide.

37. (canceled)

38. (canceled)
